(19) United States
(12) Patent Application Publication
(10) Pub. No.: US 2024/0277404 A1
(43) Pub. Date: Aug. 22, 2024

(54) ELECTRODE EDGE TRANSITION TO IMPROVE CURRENT DENSITY

(71) Applicant: Boston Scientific Scimed, Inc., Maple Grove, MN (US)

(72) Inventors: Allan Charles Shuros, St. Paul, MN (US); Andrew L. De Kock, Ham Lake, MN (US)

(21) Appl. No.: 18/419,107

(22) Filed: Jan. 22, 2024

Related U.S. Application Data
(60) Provisional application No. 63/447,608, filed on Feb. 22, 2023.

(51) Int. Cl. A61B 18/14; A61B 18/00

(52) U.S. Cl. CPC A61B 18/1492; A61B 2018/00083; A61B 2018/00267; A61B 2018/00351; A61B 2018/00577; A61B 2018/00613; A61B 2018/0072; A61B 2018/1467

(57) ABSTRACT

A catheter for ablating tissue through irreversible electroporation is disclosed. The catheter includes an elongated body having a proximal end and a distal end. The catheter also includes a first electrode spaced proximally from a second electrode along the elongated body, each of the first and second electrodes having a transition region terminating in an opposing edge. The catheter further includes an insulator disposed over the electrode in the transition region and extending between the first and second electrodes. In the transition region, an electrode thickness of each electrode decreases toward the opposing edge and an insulator thickness of the insulator correspondingly increases so as to maintain a generally constant combined thickness.

### CROSS-REFERENCE TO RELATED APPLICATIONS

This application claims the benefit of U.S. Provisional Application No. 63/447,608, entitled “ELECTRODE EDGE TRANSITION TO IMPROVE CURRENT DENSITY,” filed Feb. 22, 2023.

### TECHNICAL FIELD

The present disclosure relates to medical systems and methods for ablating tissue in a patient. More specifically, the present disclosure relates to medical systems and methods for ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radiofrequency (RF) ablation and cryoablation. In RF ablation, a probe is inserted into the patient and radiofrequency waves are transmitted through the probe to the surrounding tissue. The radiofrequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering. If the electroporation is irreversible, the affected cells are killed through apoptosis.

Irreversible electroporation can be used as a nonthermal ablation technique. In irreversible electroporation, trains of short, high voltage pulses are used to generate electric fields that are strong enough to kill cells through apoptosis. In ablation of cardiac tissue, irreversible electroporation can be a safe and effective alternative to the indiscriminate killing of thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells. There is a continuing need for improved devices and methods for performing cardiac tissue ablation through irreversible electroporation.

### SUMMARY

In Example 1 is a catheter for ablating tissue through irreversible electroporation includes an elongated body having a proximal end and a distal end. The catheter also includes a first electrode spaced proximally from a second electrode along the elongated body, each of the first and second electrodes having a transition region terminating in an opposing edge. The catheter further includes an insulator disposed over the electrode in the transition region and extending between the first and second electrodes; wherein, in the transition region, an electrode thickness of each electrode decreases toward the opposing edge and an insulator thickness of the insulator correspondingly increases so as to maintain a generally constant combined thickness.

Example 2 is the catheter of Example 1 wherein each transition region has a substantially similar shape.

Example 3 is the catheter of Example 1 wherein each transition region is configured such that when a voltage is applied to each of the electrodes, a current density in the transition region is generally constant.

Example 4 is the catheter of Example 1 wherein each electrode has a constant taper angle in the transition region.

Example 5 is the catheter of Example 1 wherein the insulator portion comprises a dielectric strength of from about 15 kV/mm to 60 kV/mm.

Example 6 is the catheter of Example 5 wherein the dielectric strength of the insulation determines the gradient of current density.

Example 7 is the catheter of Example 1 wherein the transition region condition is formed by a tapered insulation on the electrode so as to create a gradual transition current density from the insulation perform to an electrode to reduce the transition current density.

Example 8 is the catheter of Example 1 wherein the transition region comprises a combination of one or more steps, ramps, or transitions of various geometries.

Example 9 is the catheter of Example 1 further comprising a third and a fourth electrode and an insulator, the diameter of each of the third and the fourth electrode decreasing in the transition region toward the opposing edges and the insulator increasing in diameter correspondingly such that the catheter shaft is substantially isodiametric.

Example 10 is the catheter of Example 1 wherein the elongated body includes a tubular shaft having a proximal end and an opposite distal end.

Example 11 is the catheter of Example 10 wherein the elongated body further includes a plurality of splines each including a distal end portion coupled to a central hub, and a proximal end portion coupled to the tubular shaft.

Example 12 is the catheter of Example 11 wherein the first and second electrodes are disposed on one of the plurality of splines.

Example 13 is the catheter of Example 11 wherein the electrode assembly further comprises a plurality of proximal ablation electrodes located on each spline.

Example 14 is the catheter of Example 10 wherein the first and second electrode are disposed on the tubular shaft.

Example 15 is the catheter of Example 10 wherein each of the electrodes has a taper angle in the transition region of between about 20 and about 60 degrees.

In Example 16 a catheter for ablating tissue through irreversible electroporation includes an elongated body extending along a longitudinal axis and having a proximal end and a distal end. The catheter also includes a first electrode spaced proximally from a second electrode along the elongated body, each of the first and second electrodes having a transition region terminating in an opposing edge. The catheter further includes an insulator disposed over the electrode in the transition region and extending between the first and second electrodes; wherein, in the transition region, an electrode thickness of each electrode decreases toward the opposing edge and an insulator thickness of the insulator correspondingly increases so as to maintain a generally constant combined thickness.

Example 17 is the catheter of Example 16 wherein each transition region has a substantially similar shape.

Example 18 is the catheter of Example 16 wherein the transition region is configured such that when a voltage is applied to each of the electrodes, a current density in the transition region is generally constant.

Example 19 is the catheter of Example 18 wherein each electrode has a constant taper angle in the transition region.

Example 20 is the catheter of Example 16 wherein the insulator portion comprises a dielectric strength of from about 15 kV/mm to 60 kV/mm.

Example 21 is the catheter of Example 20 wherein the dielectric strength of the insulation determines the gradient of current density.

Example 22 is the catheter of Example 16 wherein the transition region condition is formed by a tapered insulation on the electrode creating a gradual transition current density from the insulation perform to an electrode to reduce the transition current density.

Example 23 is the catheter of Example 16 wherein the transition region comprises a combination of one or more steps, ramps, or transitions of various geometries.

Example 24 is the catheter of Example 16 further comprising a third and a fourth electrode and an insulator, the diameter of each of the third and the fourth electrode decreasing in the transition region toward the opposing edges and the insulator increasing in diameter correspondingly such that the catheter shaft is substantially isodiametric.

In Example 25 a catheter for ablating cardiac tissue through irreversible electroporation includes an elongated shaft extending along a longitudinal axis and having a proximal end and a distal end. The catheter also includes a tip electrode at the distal end of the elongated shaft and configured to provide pulsed field ablation signals. The catheter further includes a ring electrode located proximal of and spaced apart from the tip electrode, the first ring electrode having a distal portion. The catheter further includes an insulator disposed between the tip electrode and the first ring electrode; wherein the distal portion of the ring electrode is tapered in the distal direction along the longitudinal axis in the transition region, such that an electrode thickness of the electrode decreases and an insulator thickness of the insulator increases to maintain a generally uniform catheter diameter.

Example 26 is the catheter of Example 25 wherein the tip electrode is tapered in the distal direction along the longitudinal axis in the transition region, such that the electrode thickness decreases and the insulator thickness increases to maintain a generally uniform catheter diameter.

Example 27 is the catheter of Example 25 wherein each transition region has a substantially similar shape.

Example 28 is the catheter of Example 25 wherein the transition region is configured such that when a voltage is applied to each of the electrodes, a current density in the transition region is generally constant.

Example 29 is the catheter of Example 28 wherein each electrode has a constant taper angle in the transition region.

Example 30 is the catheter of Example 25 wherein the insulator portion comprises a dielectric strength of from about 15 kV/mm to 60 kV/mm.

Example 31 is the catheter of Example 30 wherein the dielectric strength of the insulation determines the gradient of current density.

Example 32 is the catheter of Example 25 wherein the transition region condition is formed by a tapered insulation on the electrode so as to create a gradual transition current density from the insulation perform to an electrode to reduce the transition current density.

Example 33 is the catheter of Example 25 wherein the transition region comprises a combination of one or more steps, ramps, or transitions of various geometries.

Example 34 is the catheter of Example 25 wherein the elongated shaft further comprises a third and a fourth electrode and an insulator, wherein the third electrode is tapered in the distal direction along the longitudinal axis in the transition region, such that the electrode thickness decreases and the insulator thickness increases to maintain a generally uniform catheter diameter.

Example 35 is a method of making a catheter for ablating cardiac tissue through irreversible electroporation, the method including providing an elongated shaft extending along a longitudinal axis and having a proximal end and a distal end. The method also including securing a first electrode spaced apart from a second electrode along the elongated shaft, each of the electrodes having a transition region terminating in an opposing edge. The method further including securing an insulator disposed over the electrode in the transition region and extending between the first and second electrodes, wherein the diameter of each of the first and second electrodes decreases in the transition region toward the opposing edges and the insulator increases in diameter correspondingly such that the catheter shaft is substantially isodiametric.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the subject matter of the disclosure.

FIG. 2 is an isometric illustration of a distal portion of a cardiac ablation catheter for use in the electrophysiology system of FIG. 1, in accordance with embodiments of the subject matter of the disclosure.

FIG. 3 is an isometric illustration of a distal portion of a splined catheter for use in the electrophysiology system of FIG. 1, in accordance with embodiments of the subject matter of the disclosure.

FIGS. 4A and 4B illustrate the current density generated near the edge of ablation electrodes of a traditional pulsed field ablation catheter compared to an enhanced edge transition pulsed field ablation catheter during operation.

FIGS. 5A-5D show exemplary electrode edge transition phases, in accordance with embodiments of the subject matter of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but yet still cooperate or interact with each other.

Throughout the present disclosure and in the claims, numeric terminology, such as first and second, is used in reference to various components or features. Such use is not intended to denote an ordering of the components or features. Rather, numeric terminology is used to assist the reader in identifying the component or features being referenced and should not be narrowly interpreted as providing a specific order of components or features.

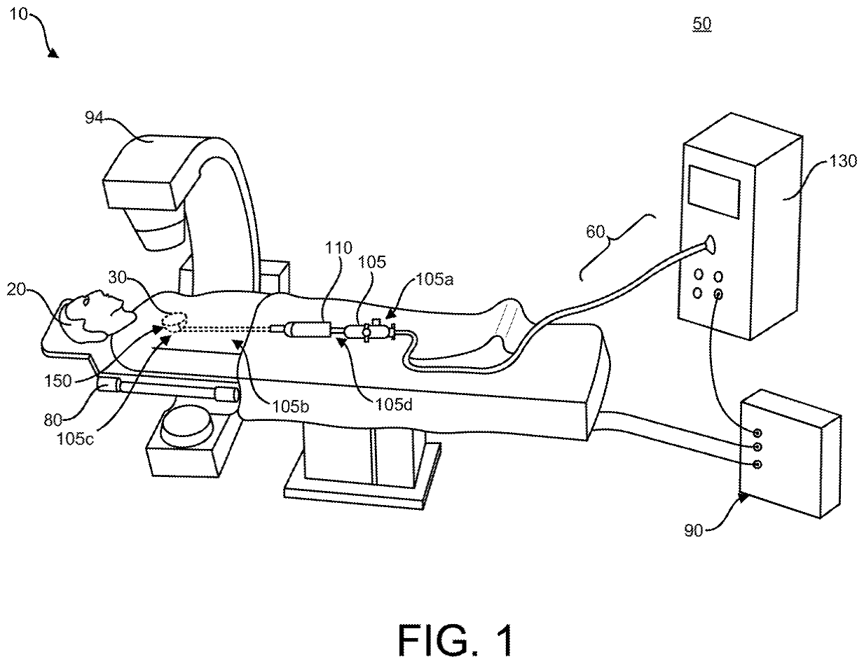

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an electroporation device 60 and an optional localization field generator 80. Also, the clinical setting 10 includes additional equipment such as imaging equipment 94 (represented by the C-arm) and various controller elements configured to allow an operator to control various aspects of the electrophysiology system 50. As will be appreciated by the skilled artisan, the clinical setting 10 may have other components and arrangements of components that are not shown in FIG. 1.

The electroporation device 60 includes a cardiac ablation catheter 105, an introducer sheath 110, a controller 90, and an electroporation generator 130. In embodiments, the electroporation device 60 is configured to deliver electric field energy to target tissue in the patient's heart 30 to create tissue apoptosis, rendering the tissue incapable of conducting electrical signals. The controller 90 is configured to control functional aspects of the electroporation device 60. In embodiments, the controller 90 is configured to control the electroporation generator 130 to generate electrical pulses, for example, the magnitude of the electrical pulses, and the timing and duration of electrical pulses. In embodiments, the electroporation generator 130 is operable as a pulse generator for generating and supplying pulse sequences to the cardiac ablation catheter 105.

In embodiments, the introducer sheath 110 is operable to provide a delivery conduit through which the cardiac ablation catheter 105 may be deployed to the specific target sites within the patient's heart 30. It will be appreciated, however, that the introducer sheath 110 is illustrated and described herein to provide context to the overall electrophysiology system 50.

In the illustrated embodiment, the cardiac ablation catheter 105 includes a handle 105a, an elongated shaft 105b, and a distal portion 150. As shown, the shaft has a distal end 105c and a proximal end 105d, and the proximal end 105d of the shaft 105b extends distally from the handle 105a. The handle 105a is configured to be operated by a user to position the distal portion 150 at the desired anatomical location. The shaft 105b generally defines a longitudinal axis of the cardiac ablation catheter 105. The shaft 105b may include a molded articulation joint for spine reinforcement and steering capability. More details may be found at U.S. Pat. App. No. 63/129,960, which is hereby incorporated by reference in its entirety.

As shown, the distal portion 150 is located at or proximate the distal end 105c of the shaft 105b. In embodiments, the distal portion 150 is electrically coupled to the electroporation generator 130, to receive electrical pulse sequences or pulse trains, thereby selectively generating electrical fields for ablating the target tissue by irreversible electroporation.

In certain embodiments, the cardiac ablation catheter 105 is a point catheter that includes a linear body toward the distal end. In embodiments, the distal portion 150 includes one or more electrodes disposed on the shaft 105b. In some implementations, the distal portion 150 includes one or more electrode pairs. In some embodiments, the distal portion 150 includes one or more ablation electrodes and one or more sensing electrodes. In certain implementations, the distal portion 150 includes a pair of ablation electrodes configured to generate electrical fields sufficient for irreversible electroporation ablation. In some examples, the ablation electrode pair including a tip electrode covering the distal end of the catheter 105 and a ring electrode disposed proximate to the tip electrode. As used herein, a ring electrode refers to an electrode having a ring shape. In some designs, the pair of ablation electrodes include two ring electrodes disposed proximate to the distal end of the catheter 105.

In embodiments, the electrode positions and sizes are specifically designed to allow flexibility. For example, the electrodes are designed to be relatively short in length. As another example, two electrodes have a relatively larger spacing to allow flexibility and/or deflection. In some examples, the one or more electrodes include one or more pairs of ablation electrodes and one or more pairs of sensing electrodes. The sensing electrodes may be used to sense electrical signals related to a patient's heart, which allows an operator or a system to determine whether ablation has occurred or not. In some designs, the electrical signals can be used to determine a location or proximate location of the cardiac ablation catheter 105. In some embodiments, other sensors, such as force sensors, navigation sensors (e.g., five or six degree-of-freedom (“DoF”) sensors), may be incorporated in the distal portion 150.

In some embodiments, the one or more sensing electrodes on the cardiac ablation catheter 105 can measure electrical signals and generate output signals that can be processed by a controller (e.g., the controller 90) to generate an electro-anatomical map. In some instances, electro-anatomical maps are generated before ablation for determining the electrical activity of the cardiac tissue within a chamber of interest. In some instances, electro-anatomical maps are generated after ablation in verifying the desired change in electrical activity of the ablated tissue and the chamber as a whole. The sensing electrodes may be used to determine the position of the catheter 105 in three-dimensional space within the body. For example, when the operator moves the catheter 105 within a cardiac chamber of a patient, the boundaries of catheter movement can be determined by the controller 90, which may include or couple to a mapping and navigation system, to form the anatomy of the chamber. The chamber anatomy may be used to facilitate navigation of the catheter 105 without the use of ionizing radiation such as with fluoroscopy, and for tagging locations of ablations as they are completed to guide spacing of ablations and aid the operator in fully ablating the anatomy of interest.

According to embodiments, various components (e.g., the controller 90) of the electrophysiological system 50 may be implemented on one or more computing devices. A computing device may include any type of computing device suitable for implementing embodiments of the disclosure. Examples of computing devices include specialized computing devices or general-purpose computing devices such as workstations, servers, laptops, portable devices, desktop, tablet computers, hand-held devices, general-purpose graphics processing units (GPGPUs), and the like, all of which are contemplated within the scope of FIG. 1 with reference to various components of the system 50.

In some embodiments, a computing device includes a bus that, directly and/or indirectly, couples the following devices: a processor, a memory, an input/output (I/O) port, an I/O component, and a power supply. Any number of additional components, different components, and/or combinations of components may also be included in the computing device. The bus represents what may be one or more busses (such as, for example, an address bus, data bus, or combination thereof). Similarly, in some embodiments, the computing device may include a number of processors, a number of memory components, a number of I/O ports, a number of I/O components, and/or a number of power supplies. Additionally, any number of these components, or combinations thereof, may be distributed and/or duplicated across a number of computing devices.

In some embodiments, the system 50 includes one or more memories (not illustrated). The one or more memories includes computer-readable media in the form of volatile and/or nonvolatile memory, transitory and/or non-transitory storage media and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In some embodiments, the one or more memories store computer-executable instructions for causing a processor (e.g., the controller 90) to implement aspects of embodiments of system components discussed herein and/or to perform aspects of embodiments of methods and procedures discussed herein.

Computer-executable instructions may include, for example, computer code, machine-useable instructions, and the like such as, for example, program components capable of being executed by one or more processors associated with a computing device. Program components may be programmed using any number of different programming environments, including various languages, development kits, frameworks, and/or the like. Some or all of the functionality contemplated herein may also, or alternatively, be implemented in hardware and/or firmware.

In some embodiments, the memory may include a data repository implemented using any one of the configurations described below. A data repository may include random access memories, flat files, XML files, and/or one or more database management systems (DBMS) executing on one or more database servers or a data center. A database management system may be a relational (RDBMS), hierarchical (HDBMS), multidimensional (MDBMS), object oriented (ODBMS or OODBMS) or object relational (ORDBMS) database management system, and the like. The data repository may be, for example, a single relational database. In some cases, the data repository may include a plurality of databases that can exchange and aggregate data by data integration process or software application. In an exemplary embodiment, at least part of the data repository may be hosted in a cloud data center. In some cases, a data repository may be hosted on a single computer, a server, a storage device, a cloud server, or the like. In some other cases, a data repository may be hosted on a series of networked computers, servers, or devices. In some cases, a data repository may be hosted on tiers of data storage devices including local, regional, and central.

Various components of the system 50 can communicate via or be coupled to via a communication interface, for example, a wired or wireless interface. The communication interface includes, but not limited to, any wired or wireless short-range and long-range communication interfaces. The wired interface can use cables, umbilicals, and the like. The short-range communication interfaces may be, for example, local area network (LAN), interfaces conforming known communications standard, such as Bluetooth® standard, IEEE 702 standards (e.g., IEEE 702.11), a ZigBee® or similar specification, such as those based on the IEEE 702.15.4 standard, or other public or proprietary wireless protocol. The long-range communication interfaces may be, for example, wide area network (WAN), cellular network interfaces, satellite communication interfaces, etc. The communication interface may be either within a private computer network, such as intranet, or on a public computer network, such as the internet.

As will be explained in greater detail elsewhere herein, the various embodiments of the present disclosure, and in particular the distal portion 150, employ novel structural features to improve the clinical performance as well as enhance the manufacturability of the ablation catheter 105. In particular, the distal portion 150 includes an insulator portion to, among other things, support and locate the tip electrode and the adjacent ring electrode, as well as operate to electrically insulate the various electrical components of the distal portion 150.

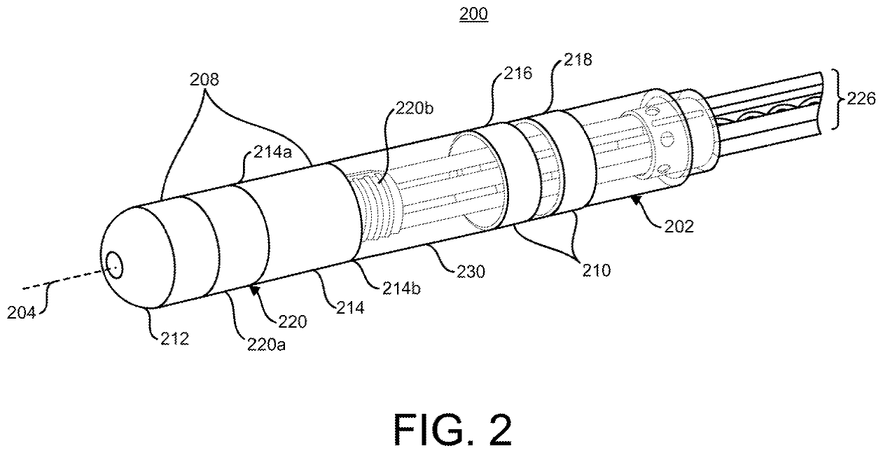

FIG. 2 is an isometric illustration of a distal portion of a cardiac ablation catheter 200. In embodiments, the cardiac ablation catheter 200 corresponds to the ablation catheter 105 depicted in FIG. 1 and includes a distal portion 202.

As shown, the distal portion 202 is disposed axially along a longitudinal axis 204 defined by the shaft (not shown in FIG. 2) of the ablation catheter 200. The distal portion 202 includes a pair of electrodes 208 including a tip electrode 212 and a ring electrode 214, the tip electrode 212 being located at the distal end of the distal portion 202, and the ring electrode 214 located proximal of and spaced apart from the tip electrode 212. As shown, the ring electrode has a distal leading end 214a and a proximal trailing end 214b. In embodiments, the distal portion 202 may include additional electrodes, e.g., an additional pair of electrodes 210 including electrodes 216, 218 disposed proximally of and longitudinally spaced from the electrodes 212 and 214. More or fewer electrodes may be employed in other embodiments within the scope of the present disclosure.

The operation of the various electrodes (or electrode pairs) can vary depending on the clinical use of the ablation catheter 200. In various embodiments, the electrodes 212, 214, 216 and 218 are configured to operate as ablation electrodes, sensing electrodes, or both. For example, any or all of the electrodes 212, 214, 216 and 218 can be configured to be operable for the delivery of ablative energy to target tissue. Additionally, or alternatively, any or all of the electrodes 212, 214, 216 and 218 can be operable as sensing electrodes configured to sense electrical signals (e.g., intrinsic cardiac activation signals and/or electric fields generated by injected currents for use in impedance-based location tracking, tissue proximity or contact sensing, and the like). In one embodiment, the pair of electrodes 208 may be configured to operate as ablation electrodes, e.g., for bi-polar delivery of ablation energy, and in particular, pulsed-field ablation energy for focal ablation of cardiac tissue. In embodiments, the electrodes 216, 218 may be operable as sensing electrodes, or alternatively, as ablation electrodes. In some instances, the second pair of electrodes 210 is configured to measure local impedance, and may act as location sensors for sensing local electric fields in 5 degrees of freedom (e.g., 5 different motions—x, y, z, acceleration, and rotation). In embodiments, except as specifically described herein, the electrodes 212, 214, 216 and 218 may be configured in accordance with those described in co-pending and commonly-assigned U.S. Pat. App. No. 63/194,716, which is hereby incorporated by reference in its entirety.

In one exemplary embodiment, the electrode pair 208 may be activated with a first polarity, and the electrode pair 210 can be activated with a second polarity opposite the first polarity, so as to define an ablation vector and corresponding electric field therebetween. It is emphasized, however, that the present disclosure is not limited to the particular electrode configurations and number of electrodes depicted in FIG. 2. Rather, the skilled artisan will appreciate that additional variations of electrode configurations, numbers of electrodes, and the like may be employed within the scope of the present disclosure.

In embodiments, as shown, the distal portion 202 includes an insulator portion 220, located between the tip electrode 212 and the ring electrode 214. In the illustrated embodiment, the insulator portion 220 includes a distal portion 220a and a proximal portion 220b (partially illustrated in FIG. 2). As further shown, the distal portion 220a of the insulator portion 220 is disposed between the tip electrode 212, and the ring electrode 214 is disposed over part of the proximal portion 220b such that the distal leading end 214a of the ring electrode 214 abuts a radial shoulder of the insulator portion 220. The insulator portion 220 includes a longitudinal interspacing along the longitudinal axis 204 in between the tip electrode 212 and the distal leading end 214a of the ring electrode 214. In embodiments, the insulator portion 220 provides an insulation layer between the conductive surfaces and wires at high potential to each other within the tip region of the catheter.

In some instances, the insulator portion provides means for routing conductive wires 226 through the distal portion 202. In some instances, the insulator portion provides positive placement features for tip components to allow for better component spacing and fitment into subsequent process steps (i.e., mold fit). In some instances, the insulator portion provides protection for components of the cardiac ablation catheter 200, such as navigational sensors or thermocouples, during various use conditions.

In various embodiments, the distal portion 202 further includes an insulating material 230 disposed at least proximally of the ring electrode 214 and encapsulating and forming an outer insulative surface of the distal portion 202. In embodiments, the insulating material 230 is disposed between the ring electrode 214 and 216. In some embodiments, the insulating material 230 is formed by an overmolding process. Alternatively, the insulating material 230 can be formed using a reflow process in which one or more tubular segments of insulating material are disposed about the partially-assembled distal portion 202 and then heated, as is known in the art. In other embodiments, the insulating material 230 is pre-molded and pre-formed. Embodiments employing an overmolding process to provide the insulating material 230 can have certain advantages, e.g., to reduce or even eliminate the need for subsequent processing (such as the injection of medical adhesive to complete the assembly process and provide a fluid-tight connections between the various components). The insulating material may be commercially available Pebax® 55D and Pelathane® 55D. Both materials may be used in an overmolding process and bonded to an “epoxy bondable” wire insulation. Pellethane may adhere to the tip insulator using primer (e.g., Sivate™ E610) and plasma. Pebax may adhere to the tip insulator using adhesive (e.g., Thermedics 1-MP) without plasma.

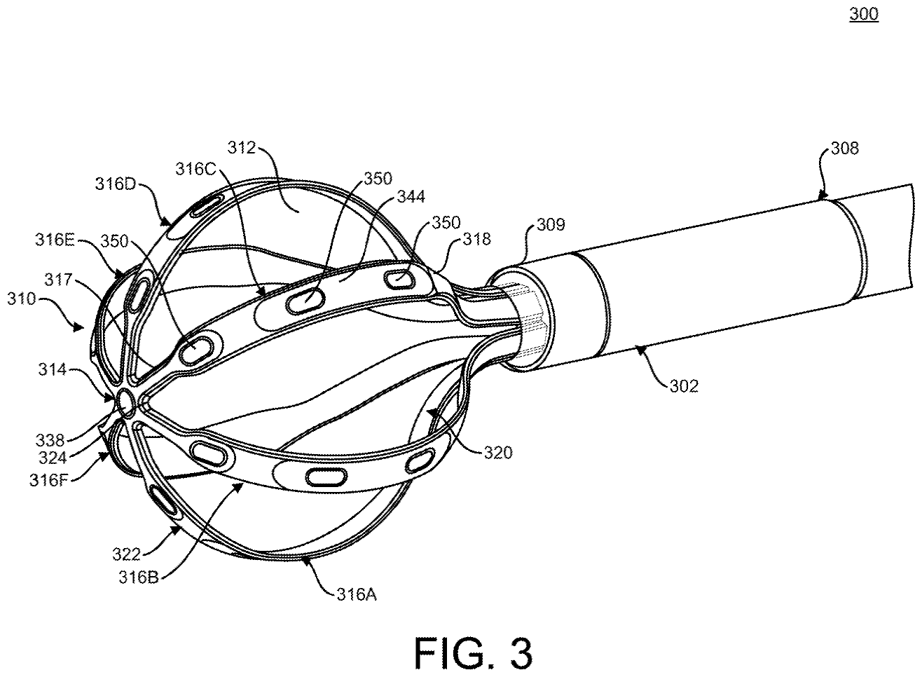

FIG. 3 is a partial perspective illustration of a cardiac ablation catheter 300 having a catheter distal portion 302 according to an embodiment of the present disclosure. In embodiments, the cardiac ablation catheter 300 corresponds to the ablation catheter 105 depicted in FIG. 1.

As shown, the ablation catheter 300 has a tubular outer shaft 308 having a shaft distal end 309, and an electrode assembly 310 extending distally from the distal end 309 of the outer shaft 308. In embodiments, the electrode assembly 310 is configured to self-expand from a collapsed configuration when constrained within a delivery sheath to a pre-defined expanded configuration defining an inner space 312. As will be explained in greater detail herein, the electrode assembly 310 comprises of multiple ablation electrodes configured to receive pulsed electrical signals from the electroporation console 130 of FIG. 1, thereby creating pulsed electric fields sufficient for ablating target tissue via irreversible electroporation. Additionally, the electrode assembly 310 further includes a plurality of mapping and sensing electrodes configured for, among other things, sensing cardiac electrical signals, localization of the electrode assembly 310 within the patient anatomy, and determining proximity to target tissue within the anatomy.

In the illustrated embodiment, the electrode assembly 310 includes a distally located central hub portion 314 and a plurality of splines 316A-316F extending proximally from the central hub portion 314. As further shown, each respective spline 316A-316F has a distal end portion 317 and a proximal end portion 318. While in FIG. 3 the distal end portion 317 and the proximal end portion 318 are only shown on the spline 316C, each of the plurality of splines 316A-316F has a distal end portion 317 and a proximal end portion 318. As shown, the proximal end portion 318 is attached to and constrained by the distal end 309 of the outer shaft 302. In embodiments, the particular geometry of the splines 316A-316F and the related components, e.g., ablation and mapping electrodes, is optimized to provide desired mechanical and therapeutic/diagnostic capabilities.

In the illustrated embodiment, the splines 316A-316F are composed of a support member 320 and a flexible circuit 322 secured to and disposed over an outer surface of the support member 320. The support member 320 functions, among other things, as a primary structural support of the electrode assembly 310, and thus primarily defines the mechanical characteristics of the electrode assembly 310. In embodiments, the support member 320 is formed from a super elastic material (metal or polymer) to provide desired mechanical/structural properties to the electrode assembly 310. In embodiments, the support member 320 is formed from a super elastic metal alloy, e.g., a nickel-titanium alloy.

The support member 320 includes a support member hub 324 and a plurality of support member branches. In embodiments, the support member branches can be selectively configured along their lengths to tune the mechanical characteristics of the electrode assembly 310. As shown, the flexible circuit 322 includes a distal ablation electrode 338. The flexible circuit 322 further includes a plurality of proximal ablation electrodes 344 and a plurality of spline sensing electrodes 350. While the proximal ablation electrodes 344 and spline sensing electrodes 350 are only shown on the spline 316C, each of the plurality of splines 316A-316F include a proximal ablation electrode 344 and a plurality of spline sensing electrodes 350. In some embodiments, insulation is used to create a surface flush with the electrode spline surface. In other embodiments, only a portion of the transition region is covered. In such embodiments, the electrode or electrodes protrude from the electrode spline surface.

In the illustrated embodiment, each of the spline sensing electrodes 350 is disposed within a periphery of one of the proximal ablation electrodes 244 or one of the radial segments of the distal ablation electrode 338. For example, as shown, each of the distal-most spline sensing electrode 350 is disposed within a periphery of a respective one of the radial segments of the distal ablation electrode 338 and is electrically isolated from the distal ablation electrode 338. Additionally, a plurality of the more proximally located spline sensing electrodes 350 are disposed along and within a periphery of a respective one of each of the proximal ablation electrodes 344 and electrically isolated therefrom.

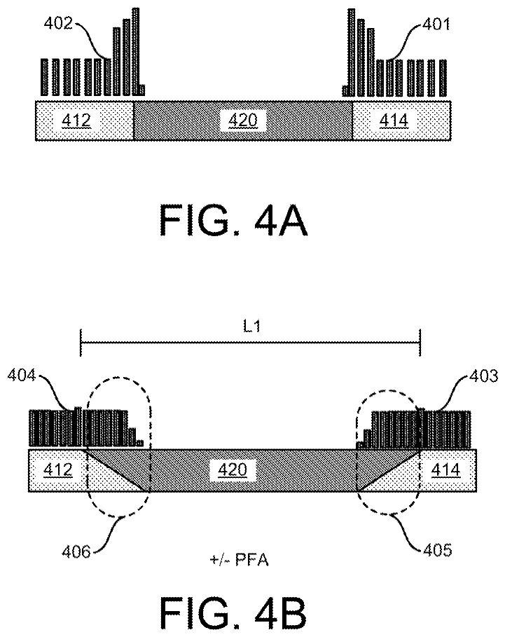

FIGS. 4A and 4B show the current density generated near the edge of the ablation electrodes of a traditional pulsed field ablation catheter compared to an enhanced edge transition pulsed field ablation catheter during operation. The current density that is generated and described in FIGS. 4A and 4B are on the exterior portion of the electrodes and only display one-half of the cross-section of the cardiac ablation catheter 200 of FIG. 2. As shown, the first electrode 414, insulator portion 420, and second electrode 412 correspond with the ring electrode 214, insulator portion 220, and tip electrode 212 of FIG. 2, according to embodiments of the present disclosure.

As shown in FIG. 4A, the conventional pulsed field ablation design produces an ablative current density 401 when a voltage is applied to the first electrode 414. At the intersection of the electrode and the insulator portion 420, the current density 401 increases significantly, for example, in some cases the current density about doubles. As shown, conventional pulsed field ablation electrodes generate high current density peaks at the electrode edges. The “edge” or “electrode edge” refers to the transition area between a metal electrode and an insulator portion. These high current peaks, as illustrated in the ablative current density 401 and 402, can induce arcing. Electrical arcing can cause various problems, including thermal damage, gas embolization, and ineffective ablation energy delivery, among other issues.

As shown in FIG. 4B, the enhanced edge transition pulsed field ablation design produces a more constant current density 403 and 404. As shown, each facing electrode pair includes opposing edges. The first electrode 414 is spaced apart from the second electrode 412, each of the electrodes having a transition region terminating in an opposing edge. The insulator portion 420 is disposed over the electrode in the transition region and extends between the first and second electrodes 414 and 412. As shown, unlike the conventional transition from electrode to insulator in FIG. 4A, the enhanced edge transition design contains a gradual transition zone 405 and 406. In the transition zone 405 and 406, the diameter (or thickness) of the electrode gradually decreases in the direction toward the opposing edge, while the thickness of the insulation increases in a complementary fashion, such that the combination of electrode and insulation results in a substantially isodiametric structure. In embodiments, the transition zone 405 and 406 may be made of different insulative materials than the rest of the insulator portion 420. For example, the transition zone 405 and 406 may be made of semi-conductive material while the rest of the insulator portion 420 may be fully insulative.

As shown in FIG. 4B, the transition zones 405 and 406 are defined by the interspacing length L1. The interspacing length L1 defines a longitudinal region in between the first electrode 414 and the second electrode 412. As the length of L1 is reduced, the current density generated on the ablation electrodes in the transition zone 405 and 406 increase. As shown, zones 405 and 406 create a transition region where the thickness (or diameter) of the electrode decreases and the thickness of the insulation layer increases. This transition region mitigates the current density peaks seen in conventional designs. In various embodiments, the current density produced at the electrode and in the transition region is generally constant. The optimal angle of the tapered transition zone 405 and 406 is one that avoids the current density spikes seen in FIG. 4A. In embodiments, the taper angle in the transition zone 405 and 406 is between 20 and 60 degrees and, in other embodiments, the taper angle in the transition zone is between 30 degrees and 45 degrees.

As shown in transition zone 406, the current density 404 gradually increases from the electrode edge, which is mostly insulated, to the full electrode, which is un-insulated. Additionally, the thickness of the insulator portion 420 gradually decreases from 100% to 0%. Thus, the enhanced gradual edge transition design reduces the current density that is generated on the ablative electrodes in the transition zones. This enhanced design eliminates the large current density peaks created in the traditional pulsed field ablation design, and thus eliminating the risk of electrical arcing, sparks, localized heating, electrolysis gas production, among many other issues.

In embodiments, electrode wall thickness can range from 0.0001 inches, like in the cardiac ablation catheter 300 of FIGS. 3, to 0.010 inches for larger electrodes. In further embodiments, assuming a 0.006-inch (0.15 mm) electrode wall thickness, the full thickness insulation at the electrode edge can withstand 3 kV—exceeding the maximum voltage of most pulsed field ablation energy applications for cardiac ablation. The dielectric strength of the insulation determines the gradient of current density. The dielectric strength can range from about 15 kV/mm to 60 kV/mm. In embodiments, the insulating material can include polyurethane, PEBAX®, polyether ether ketone (PEEK), polycarbonate, Isoplast®, among others.

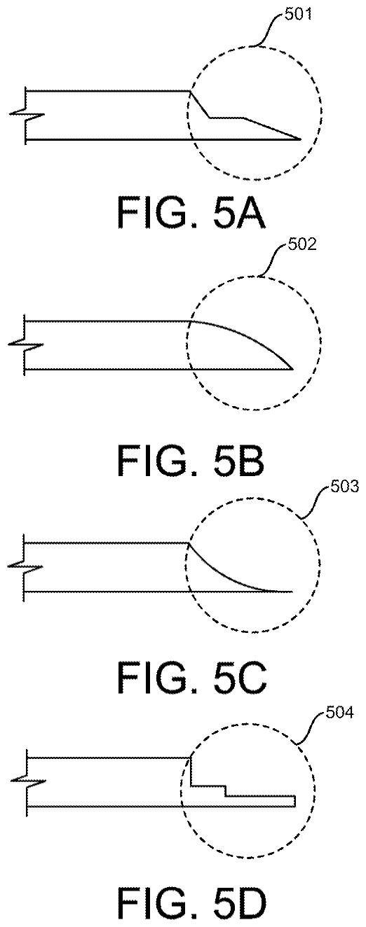

FIGS. 5A-5D demonstrates four other exemplary electrode edge transition phases, in accordance with embodiments of the subject matter of the disclosure. FIGS. 5A-5D demonstrate one-half of an electrode edge transition portion. As shown, ideal transition may not be completely linear. FIG. 5A demonstrates a stepped trapezoidal current density electrode edge transition portion 501. FIG. 5B demonstrates an outward curved current density electrode edge transition portion 502. FIG. 5C demonstrates an inward curved current density electrode edge transition portion 503. FIG. 5D demonstrates a multistep current density electrode edge transition portion 504. Thus, a combination of one or more steps, ramps, or transitions of various geometries may be desired to achieve maximum function.

In the various embodiments described above, a number of techniques may be used to add the insulation in the transition regions, including for example, overmolding, reflow, lamination overlay, fluoromasking or laminating layers with adhesives. Additionally, the electrode surface may be textured at transition to increase surface area and improve insulation bonding. In some instances, the electrode surface may be treated with a primer or other binding agents to improve insulation bonding. In embodiments, electrode edges are rounded or radiused. The interior edges may be broken or radiused to prevent mechanical damage from bending during device manufacturing and function.

In embodiments, conductive polymers may be used in the insulation transition regions, gradually changing resistivity. This may be done in addition to, or instead of, the tapered wall thickness of the electrodes or insulation material previously mentioned above. These polymers include, but are not limited to, polyacetylene, polypyrrole, polyindole polyaniline, and their copolymers, and Poly(p-phenylene vinylene) (PPV) and its soluble derivatives. These polymers have the ability to be tuned for resistivity and may serve better than typical insulative polymers. Additionally, in instances, to counteract possible high current density at thinner insulation areas, a conductive polymer may be applied in the transition regions.

Note that while the embodiments above are described with respect to a bipolar ablation configuration, the concepts described could also be applied in a monopolar ablation configuration. In such a configuration, for example, monopolar ablation electrodes could extend from an electrode on the catheter to a return electrode positioned outside the heart, for example an external electrode placed on the patient's skin.

Embodiments of the present disclosure provide systems, devices, and methods for selective and rapid application of pulsed electric fields to ablate tissue by irreversible electroporation. Generally, the systems, devices, and methods described herein may be used to generate large electric field magnitudes at desired regions of interest and reduce peak electric field values elsewhere in order to reduce unnecessary tissue damage and electrical arcing. An irreversible electroporation system as described herein may include a signal generator and a processor configured to apply one or more voltage pulse waveforms to a selected set of electrodes of an ablation device to deliver energy to a region of interest (e.g., ablation energy for a set of tissue in a pulmonary vein ostium or antrum). The pulse waveforms disclosed herein may aid in therapeutic treatment of a variety of cardiac arrhythmias (e.g., atrial fibrillation). In order to deliver the pulse waveforms generated by the signal generator, one or more electrodes of the ablation device may have an insulated electrical lead configured for sustaining a voltage potential in the order of several hundred volts to several thousand volts. The electrodes may be independently addressable such that each electrode may be controlled (e.g., deliver energy) independently of any other electrode of the device. In this manner, the electrodes may deliver different energy waveforms with different timing synergistically for electroporation of tissue.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A catheter for ablating tissue through irreversible electroporation, the catheter comprising:
an elongated body extending along a longitudinal axis and having a proximal end and a distal end;
a first electrode spaced proximally from a second electrode along the elongated body, each of the first and second electrodes having a transition region terminating in an opposing edge; and
an insulator disposed over the electrode in the transition region and extending between the first and second electrodes,
wherein, in the transition region, an electrode thickness of each electrode decreases toward the opposing edge and an insulator thickness of the insulator correspondingly increases so as to maintain a generally constant combined thickness.

2. The ablation catheter of claim 1, wherein each transition region has a substantially similar shape.

3. The ablation catheter of claim 1, wherein the transition region is configured such that when a voltage is applied to each of the electrodes, a current density in the transition region is generally constant.

4. The ablation catheter of claim 2, wherein each electrode has a constant taper angle in the transition region.

5. The ablation catheter of claim 1, wherein the insulator portion comprises a dielectric strength of from about 15 kV/mm to 60 kV/mm.

6. The ablation catheter of claim 5, wherein the dielectric strength of the insulation determines the gradient of current density.

7. The ablation catheter of claim 1, wherein the transition region condition is formed by a tapered insulation on the electrode creating a gradual transition current density from the insulation perform to an electrode to reduce the transition current density.

8. The ablation catheter of claim 1, wherein the transition region comprises a combination of one or more steps, ramps, or transitions of various geometries.

9. The ablation catheter of claim 1, further comprising a third and a fourth electrode and an insulator, the diameter of each of the third and the fourth electrode decreasing in the transition region toward the opposing edges and the insulator increasing in diameter correspondingly such that the catheter shaft is substantially isodiametric.

10. A catheter for ablating cardiac tissue through irreversible electroporation, the catheter comprising:
an elongated shaft extending along a longitudinal axis and having a proximal end and a distal end;
a tip electrode at the distal end of the elongated shaft and configured to provide pulsed field ablation signals;
a ring electrode located proximal of and spaced apart from the tip electrode, the first ring electrode having a distal portion; and
an insulator disposed between the tip electrode and the first ring electrode,
wherein the distal portion of the ring electrode is tapered in the distal direction along the longitudinal axis in the transition region, such that an electrode thickness of the electrode decreases and an insulator thickness of the insulator increases to maintain a generally uniform catheter diameter.

11. The cardiac ablation catheter of claim 10, wherein the tip electrode is tapered in the distal direction along the longitudinal axis in the transition region, such that the electrode thickness decreases and the insulator thickness increases to maintain a generally uniform catheter diameter.

12. The cardiac ablation catheter of claim 10, wherein each transition region has a substantially similar shape.

13. The cardiac ablation catheter of claim 10, wherein the transition region is configured such that when a voltage is applied to each of the electrodes, a current density in the transition region is generally constant.

14. The cardiac ablation catheter of claim 13, wherein each electrode has a constant taper angle in the transition region.

15. The cardiac ablation catheter of claim 10, wherein the insulator portion comprises a dielectric strength of from about 15 kV/mm to 60 kV/mm.

16. The cardiac ablation catheter of claim 15, wherein the dielectric strength of the insulation determines the gradient of current density.

17. The cardiac ablation catheter of claim 10, wherein the transition region condition is formed by a tapered insulation on the electrode so as to create a gradual transition current density from the insulation perform to an electrode to reduce the transition current density.

18. The cardiac ablation catheter of claim 10, wherein the transition region comprises a combination of one or more steps, ramps, or transitions of various geometries.

19. The cardiac ablation catheter of claim 10, wherein the elongated shaft further comprises a third and a fourth electrode and an insulator, wherein the third electrode is tapered in the distal direction along the longitudinal axis in the transition region, such that the electrode thickness decreases and the insulator thickness increases to maintain a generally uniform catheter diameter.

20. A method of making a catheter for ablating cardiac tissue through irreversible electroporation, the method comprising:
providing an elongated shaft extending along a longitudinal axis and having a proximal end and a distal end;
securing a first electrode spaced apart from a second electrode along the elongated shaft, each of the electrodes having a transition region terminating in an opposing edge; and
securing an insulator disposed over the electrode in the transition region and extending between the first and second electrodes, wherein the diameter of each of the first and second electrodes decreases in the transition region toward the opposing edges and the insulator increases in diameter correspondingly such that the catheter shaft is substantially isodiametric.
